(19) United States
(12) Patent Application Publication
(10) Pub. No.: US 2026/0204148 A1
(43) Pub. Date: Jul. 16, 2026

(54) ARTIFICIAL INTELLIGENCE-BASED INFANT LANGUAGE TRANSLATION SYSTEM

(71) Applicant: Gregory V. Gullo, JR., Swedesboro, NJ (US)

(72) Inventor: Gregory V. Gullo, JR., Swedesboro, NJ (US)

(21) Appl. No.: 19/016,074

(22) Filed: Jan. 10, 2025

(51) Int. Cl. G08B 21/02; A61B 5/024; A61B 5/11; A61B 5/145; G10L 15/22

(52) U.S. Cl. CPC G08B 21/0211; A61B 5/024; A61B 5/11; A61B 5/14542; G10L 15/22; G10L 2015/226

(57) ABSTRACT

A computer-implemented method may include receiving at least one audio signal; receiving movement data; receiving at least one of a heart rate or an oxygen level data; receiving at least one bodily-need data; correlating, via an artificial intelligence (AI) model, the at least one bodily-need data to the at least one audio signal, the movement data, and the at least one of the heart rate or the oxygen level data; generating, via the AI model, a predictive model based on the correlating; receiving at least one second audio signal or second movement data; predicting, via the predictive model, at least one second bodily-need based on the at least one second audio signal or second movement data; and communicating a notification to a user device.

### TECHNICAL FIELD

The embodiments generally relate to the technical field of artificial intelligence-based (AI) audio-signal translation systems.

### BACKGROUND

There are few unique offerings for identifying a young child or infant's needs based on non-verbal audio cues, e.g., crying, fussing, etc. Parents and caretakers are often left to intuition and routine to determine whether an infant may need food, a nap, soothing, etc. when a child is fussy or crying.

Technological solutions may monitor the biometrics of infants. However, biometrics do not intuitively correlate to the bodily needs of infants. For example, an infant's heart rate and oxygen levels may not be a strong indication of a bodily-need, depending on a parent's understanding of heart rate and oxygen levels. In this way, parents and caretakers are, again, left to intuition and routine to determine an infant's needs.

### SUMMARY

This summary is provided to introduce a variety of concepts in a simplified form that is further disclosed in the detailed description of the embodiments. This summary is not intended to identify key or essential inventive concepts of the claimed subject matter, nor is it intended to determine the scope of the claimed subject matter.

In one aspect, the present invention is an AI-based infant language translation system configured for collecting and processing both passive and active audio or biometric data from a wearable device with built-in voice-activated microphone and pulse oximeter as well as manual data collection via recording button to determine what infant vocalizations generally mean and what needs the infant has when combined with vital signs.

In one aspect, a sensor module is configured to passively collect audio data, movement data, heart rate data, and oxygen data of an infant via a small wearable device. In one aspect, the sensor module receives bodily-need data via user input.

In one aspect, an AI module is configured to correlate the audio data, movement data, heart rate data, and oxygen data of the infant to the bodily-need data, and, based on the correlation, generate a predictive model for future bodily-needs of the infant for future audio data, movement data, heart rate data, and oxygen data.

In one aspect, a predictive module is configured to predict a second bodily-need of the infant based on a second audio data or a second movement data.

In some aspects, the system generates a notification to a user device including the second bodily-need such that a user may take action to address the second bodily-need.

Other illustrative variations within the scope of the invention will become apparent from the detailed description provided hereinafter. The detailed description and enumerated variations, while disclosing optional variations, are intended for purposes of illustration only and are not intended to limit the scope of the invention.

### BRIEF DESCRIPTION OF THE DRAWINGS

A more complete understanding of the embodiments, and the attendant advantages and features thereof, will be more readily understood by references to the following detailed description when considered in conjunction with the accompanying drawings wherein:

FIG. 1 illustrates a system architecture diagram, according to some embodiments;

FIG. 2 illustrates an application program and modules in communication with the computing system, according to some embodiments;

FIG. 3 illustrates a block diagram of an AI-based infant language translation system, according to some embodiments; and

FIG. 4 illustrates a flowchart of an AI-based infant language translation method, according to some embodiments.

### DETAILED DESCRIPTION

The specific details of the single embodiment or variety of embodiments described herein are set forth in this application. Any specific details of the embodiments described herein are used for demonstration purposes only, and no unnecessary limitation(s) or inference(s) are to be understood or imputed therefrom.

Before describing exemplary embodiments in detail, it is noted that the embodiments reside primarily in combinations of components related to devices and systems. Accordingly, the device components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

The disclosed system combines a wearable device that automatically, passively records an infant's vocalizations and processes via AI to detect commonalities between certain sounds and an associated heart rate and oxygen level measured, for example, via a wearable device. In embodiments, the wearable device is a soft device that may be secured around an infant's limb or torso and may be wirelessly or charged via wired connection. The user of a software application then enters data on basic bodily needs such as hunger, diaper change, discomfort, need for attention, soothing, etc. An AI module is configured to correlate the infant's needs as entered by the user with the recorded vocalizations and vital signs to learn what vocalizations are associated with bodily-needs.

In this way, the disclosed system overcomes shortcomings of conventional systems that may monitor only vocalizations or biometrics but do not monitor both simultaneously and do not correlate vocalization to biometrics via AI.

In embodiments, the disclosed system may include a wearable device configured to include a silicone wristband or a cotton and hook-and-loop wristband with a pulse oximeter feature configured to be held against the skin on the underside of the wristband. The wearable device may include an integrated microphone on the outside of that same electronic feature opposite the pulse oximeter feature. All parts of the device would be soft to not injury the infant if they were to hit themselves inadvertently with the device. The system may include a software application that is usable via a computing device, such as a smartphone or smartwatch, and which is in operable communication with the wearable device over a network or other wireless communication protocol.

In embodiments, the disclosed system may be configured to facilitate identifying certain vocal sounds that are directly correlated to certain illnesses such as respiratory illnesses that cause certain breathing sounds in infants. This has the potential to, when combined with artificial intelligence enhancements, save lives with early detection of disease that can be caught early when compared via AI to previous recordings of sounds associated with certain illnesses.

Implementations of the invention involve the technical field of AI-based audio and biometrics correlation, including the steps of correlating, via an AI model, at least one bodily-need data to the at least one audio signal, the movement data, and the at least one of the heart rate or the oxygen level data; generating, via the AI model, a predictive model based on the correlating; and predicting, via the predictive model, at least one second bodily-need based on the at least one second audio signal or second movement data, and are therefore necessarily rooted in computer technology. For example, the aforementioned steps are inherently computer-based and cannot be performed in the human mind because they employ artificial intelligence and computer-based predictive modeling. The present invention amounts to more than merely implementing the generic computer as a tool to gather, analyze, and output data because the steps of the present method, system, or product improve the field of artificial intelligence-based (AI) audio-signal translation systems. Additionally, the steps of the present invention would be impossible to accomplish on pen and paper due to the volume of data being communicated and received over a network in real-time. In particular, the speed at which the steps of the present invention occur to effectuate the disclosed method, system, or product would involve large-scale, continuous wireless communication of such data. That is, the steps of the present method, system, or product are impossible to accomplish on pen and paper, cannot be accomplished as a method of organizing human activity, and amount to significantly more than merely gathering, analyzing, and outputting data.

Implementations of the present invention include implementing (executing, running, or deploying) one or more artificial intelligence models on a computing device wherein the computing device executes the artificial intelligence model's algorithms and mathematical functions on computer hardware using machine learning libraries. The computing device implements the artificial intelligence model when it performs tasks like training, making predictions, applying the model to data, decision-making, classification, or generating outputs based on inputs. In particular, the speed at which an artificial intelligence model occur to analyzes and transforms data to effectuate the disclosed method, system, or product would involve large-scale, continuous transformation of such data. As such, the present invention would be impossible to accomplish on pen and paper or in the human mind due to the volume of data being analyzed and transformed by the artificial intelligence model.

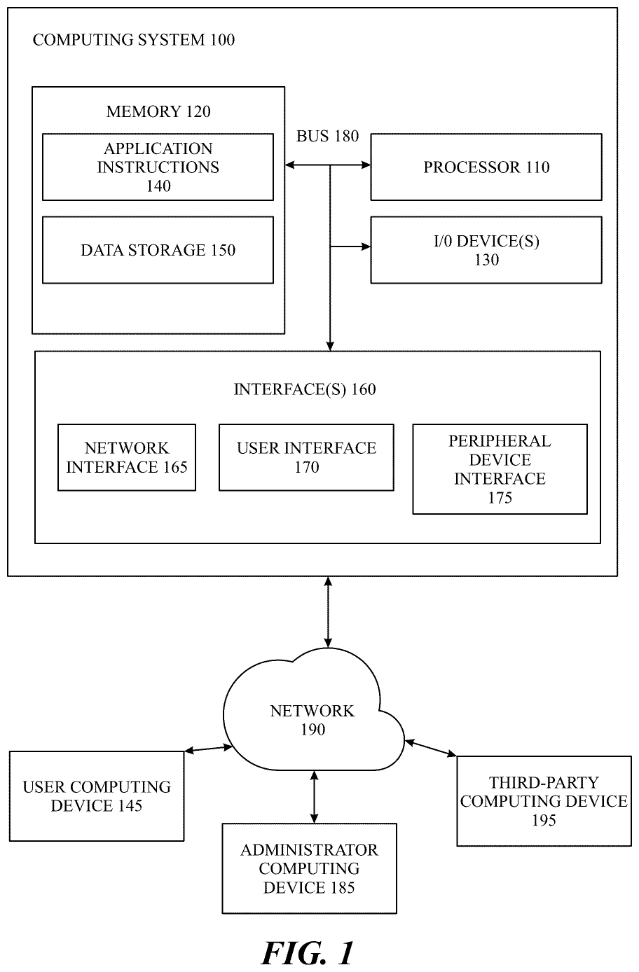

FIG. 1 illustrates an example of a computer system 100 that may be utilized to execute various procedures, including the processes described herein. The computer system 100 comprises a standalone computer or mobile computing device, a mainframe computer system, a workstation, a network computer, a desktop computer, a laptop, or the like. The computer system 100 can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive).

In some embodiments, the computer system 100 includes one or more processors 110 coupled to a memory 120 through a system bus 180 that couples various system components, such as an input/output (I/O) devices 130, to the processors 110. The bus 180 may be any of several types of bus structures including a memory bus or memory controller, a peripheral bus, and a local bus using any of a variety of bus architectures. For example, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, also known as Mezzanine bus.

In some embodiments, the computer system 100 includes one or more input/output (I/O) devices 130, such as video device(s) (e.g., a camera), audio device(s), and display(s) are in operable communication with the computer system 100. In some embodiments, similar I/O devices 130 may be separate from the computer system 100 and may interact with one or more nodes of the computer system 100 through a wired or wireless connection, such as over a network interface.

Processors 110 suitable for the execution of computer readable program instructions include both general and special purpose microprocessors and any one or more processors of any digital computing device. For example, each processor 110 may be a single processing unit or a number of processing units and may include single or multiple computing units or multiple processing cores. The processor(s) 110 can be implemented as one or more microprocessors, microcomputers, microcontrollers, digital signal processors, central processing units, state machines, logic circuitries, and/or any devices that manipulate signals based on operational instructions. For example, the processor(s) 110 may be one or more hardware processors and/or logic circuits of any suitable type specifically programmed or configured to execute the algorithms and processes described herein. The processor(s) 110 can be configured to fetch and execute computer readable program instructions stored in the computer-readable media, which can program the processor(s) 110 to perform the functions described herein.

In this disclosure, the term “processor” can refer to substantially any computing processing unit or device, including single-core processors, single-processors with software multithreading execution capability, multi-core processors, multi-core processors with software multithreading execution capability, multi-core processors with hardware multithread technology, parallel platforms, and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures, such as molecular and quantum-dot based transistors, switches, and gates, to optimize space usage or enhance performance of user equipment. A processor can also be implemented as a combination of computing processing units.

In some embodiments, the memory 120 includes computer-readable application instructions 140, configured to implement certain embodiments described herein, and a database 150, comprising various data accessible by the application instructions 140. In some embodiments, the application instructions 140 include software elements corresponding to one or more of the various embodiments described herein. For example, application instructions 140 may be implemented in various embodiments using any desired programming language, scripting language, or combination of programming and/or scripting languages (e.g., Android, C, C++, C #, JAVA, JAVASCRIPT, PERL, etc.).

In this disclosure, terms “store,” “storage,” “data store,” data storage,” “database,” and substantially any other information storage component relevant to operation and functionality of a component are utilized to refer to “memory components,” which are entities embodied in a “memory,” or components comprising a memory. Those skilled in the art would appreciate that the memory and/or memory components described herein can be volatile memory, nonvolatile memory, or both volatile and nonvolatile memory. Nonvolatile memory can include, for example, read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include, for example, RAM, which can act as external cache memory. The memory and/or memory components of the systems or computer-implemented methods can include the foregoing or other suitable types of memory.

Generally, a computing device will also include or be operatively coupled to receive data from or transfer data to, or both, one or more mass data storage devices; however, a computing device need not have such devices. The computer readable storage medium (or media) can be a tangible device that can retain and store instructions for use by an instruction execution device. The computer readable storage medium can be, for example, an electronic storage device, a magnetic storage device, an optical storage device, an electromagnetic storage device, a semiconductor storage device, or any suitable combination of the foregoing. A non-exhaustive list of more specific examples of the computer readable storage medium can include: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. In this disclosure, a computer readable storage medium is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

In some embodiments, the steps and actions of the application instructions 140 described herein are embodied directly in hardware, in a software module executed by a processor, or in a combination of the two. A software module may reside in RAM, flash memory, ROM memory, EPROM memory, EEPROM memory, registers, a hard disk, a removable disk, a CD-ROM, or any other form of storage medium known in the art. An exemplary storage medium may be coupled to the processor 110 such that the processor 110 can read information from, and write information to, the storage medium. In the alternative, the storage medium may be integrated into the processor 110. Further, in some embodiments, the processor 110 and the storage medium may reside in an Application Specific Integrated Circuit (ASIC). In the alternative, the processor and the storage medium may reside as discrete components in a computing device. Additionally, in some embodiments, the events or actions of a method or algorithm may reside as one or any combination or set of codes and instructions on a machine-readable medium or computer-readable medium, which may be incorporated into a computer program product.

In some embodiments, the application instructions 140 for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The application instructions 140 can execute entirely on the user's computer, partly on the user's computer, as a stand-alone software package, partly on the user's computer and partly on a remote computer, or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

In some embodiments, the application instructions 140 can be downloaded to a computing/processing device from a computer readable storage medium, or to an external computer or external storage device via a network 190. A network adapter card or network interface in each computing/processing device receives computer readable program instructions from the network and forwards the computer readable application instructions 140 for storage in a computer readable storage medium within the respective computing/processing device.

In some embodiments, the computer system 100 includes one or more interfaces 160 that allow the computer system 100 to interact with other systems, devices, or computing environments. In some embodiments, the computer system 100 comprises a network interface 165 to communicate with a network 190. In some embodiments, the network interface 165 is configured to allow data to be exchanged between the computer system 100 and other devices attached to the network 190, such as other computer systems, or between nodes of the computer system 100. In various embodiments, the network interface 165 may support communication via wired or wireless general data networks, such as any suitable type of Ethernet network, for example, via telecommunications/telephony networks such as analog voice networks or digital fiber communications networks, via storage area networks such as Fiber Channel SANs, or via any other suitable type of network and/or protocol. Other interfaces include the user interface 170 and the peripheral device interface 175.

In some embodiments, the network 190 corresponds to a local area network (LAN), wide area network (WAN), the Internet, a direct peer-to-peer network (e.g., device to device Wi-Fi, Bluetooth, etc.), and/or an indirect peer-to-peer network (e.g., devices communicating through a server, router, or other network device). The network 190 can comprise copper transmission cables, optical transmission fibers, wireless transmission, routers, firewalls, switches, gateway computers and/or edge servers. The network 190 can represent a single network or multiple networks. In some embodiments, the network 190 used by the various devices of the computer system 100 is selected based on the proximity of the devices to one another or some other factor. For example, when a first user device and second user device are near each other (e.g., within a threshold distance, within direct communication range, etc.), the first user device may exchange data using a direct peer-to-peer network. But when the first user device and the second user device are not near each other, the first user device and the second user device may exchange data using a peer-to-peer network (e.g., the Internet). The Internet refers to the specific collection of networks and routers communicating using an Internet Protocol (“IP”) including higher level protocols, such as Transmission Control Protocol/Internet Protocol (“TCP/IP”) or the Uniform Datagram Packet/Internet Protocol (“UDP/IP”).

Any connection between the components of the system may be associated with a computer-readable medium. For example, if software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. As used herein, the terms “disk” and “disc” include compact disc (CD), laser disc, optical disc, digital versatile disc (DVD), floppy disk, and Blu-ray disc; in which “disks” usually reproduce data magnetically, and “discs” usually reproduce data optically with lasers. Combinations of the above should also be included within the scope of computer-readable media. In some embodiments, the computer-readable media includes volatile and nonvolatile memory and/or removable and non-removable media implemented in any type of technology for storage of information, such as computer-readable instructions, data structures, program modules, or other data. Such computer-readable media may include RAM, ROM, EEPROM, flash memory or other memory technology, optical storage, solid state storage, magnetic tape, magnetic disk storage, RAID storage systems, storage arrays, network attached storage, storage area networks, cloud storage, or any other medium that can be used to store the desired information and that can be accessed by a computing device. Depending on the configuration of the computing device, the computer-readable media may be a type of computer-readable storage media and/or a tangible non-transitory media to the extent that when mentioned, non-transitory computer-readable media exclude media such as energy, carrier signals, electromagnetic waves, and signals per se.

In some embodiments, the system is world-wide-web (www) based, and the network server is a web server delivering HTML, XML, etc., web pages to the computing devices. In other embodiments, a client-server architecture may be implemented, in which a network server executes enterprise and custom software, exchanging data with custom client applications running on the computing device.

In some embodiments, the system can also be implemented in cloud computing environments. In this context, “cloud computing” refers to a model for enabling ubiquitous, convenient, on-demand network access to a shared pool of configurable computing resources (e.g., networks, servers, storage, applications, and services) that can be rapidly provisioned via virtualization and released with minimal management effort or service provider interaction, and then scaled accordingly. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

As used herein, the term “add-on” (or “plug-in”) refers to computing instructions configured to extend the functionality of a computer program, where the add-on is developed specifically for the computer program. The term “add-on data” refers to data included with, generated by, or organized by an add-on. Computer programs can include computing instructions, or an application programming interface (API) configured for communication between the computer program and an add-on. For example, a computer program can be configured to look in a specific directory for add-ons developed for the specific computer program. To add an add-on to a computer program, for example, a user can download the add-on from a website and install the add-on in an appropriate directory on the user's computer.

In some embodiments, the computer system 100 may include a user computing device 145, an administrator computing device 185 and a third-party computing device 195 each in communication via the network 190. The user computing device 145 may be utilized by a user to interact with the various functionalities of the system. The administrator computing device 185 is utilized by an administrative user to moderate content and to perform other administrative functions. The third-party computing device 195 may be utilized by third parties to receive communications from the user computing device, transmit communications to the user via the network, and otherwise interact with the various functionalities of the system.

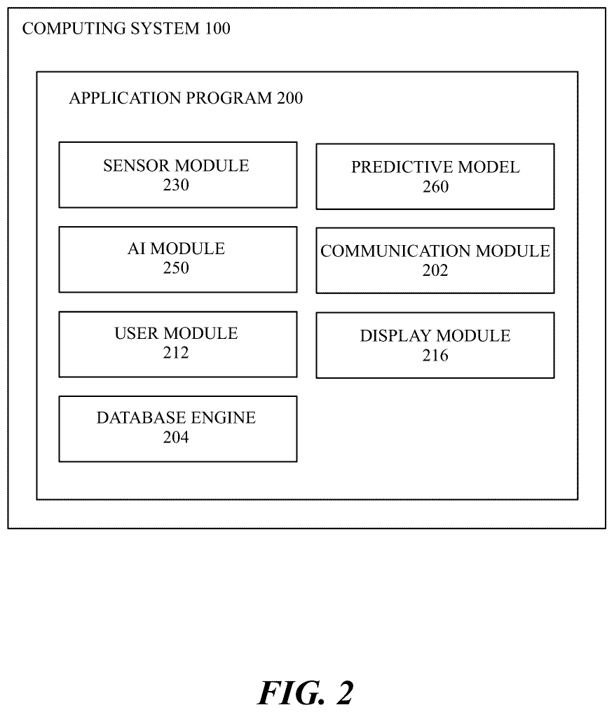

FIG. 2 illustrates an example computer architecture for the application program 200 operated via the computing system 100. The computer system 100 comprises several modules and engines configured to execute the functionalities of the application program 200, and a database engine 204 configured to facilitate how data is stored and managed in one or more databases. In particular, FIG. 2 is a block diagram showing the modules and engines needed to perform specific tasks within the application program 200.

Referring to FIG. 2, the computing system 100 operating the application program 200 comprises one or more modules having the necessary routines and data structures for performing specific tasks, and one or more engines configured to determine how the platform manages and manipulates data. In some embodiments, the application program 200 comprises one or more of a sensor module 230, a predictive module 260, an AI module 250, a communication module 202, a database engine 204, a user module 212, and a display module 216.

In some embodiments, the sensor module 230 is configured to passively collect audio data, movement data, heart rate data, and oxygen data of an infant via a small wearable device worn by, or positioned nearby an infant. The sensor module 230 may include, for example, a microphone that may be integrated into a wearable device. In one aspect, the sensor module 230 receives bodily-need data via user input through the user module 212 via application program 200, which may be an extension on a user device such as a smartphone, and which may also be operating on computing system 100, which may be, for example, a server. The sensor module 230 may include a heart rate monitor, a gyroscope, an oxygen sensor, etc., and other components suitable for measuring infant biometrics and audio data.

In some embodiments, the AI module 250 is configured to correlate audio data, movement data, heart rate data, and oxygen data also collected from the sensor module 230 to bodily-need data input by a user via the user module 212. Bodily-need data may be entered, for example, via the user computing device 145 of FIG. 1, and may include, for example, “45 minute nap at 8:30 A.M.” or “6 ounces of warm baby formula at 2 A.M.” The AI module 250 may correlate the bodily-need data to the audio data, movement data, heart rate data, and oxygen data over time and generate a predictive model based on the correlated data. The predictive model may be generated, for example, by analyzing historical bodily-need data and collected sensor module 230 data to make informed predictions about future bodily-needs. The AI module 150 may preprocess data by cleaning, normalizing, and transforming it into a format suitable for analysis. Using statistical algorithms or machine learning techniques, the AI module 150 may identify patterns and relationships within the data. These patterns form the basis for the predictive model, which the AI module 150 trains and tests to ensure accuracy. Once validated, the predictive model generates predictions by applying its learned patterns to new audio data, movement data, heart rate data, and oxygen data. The AI module 250 may also be configured to optimize the predictive model's performance by tuning parameters and evaluating results to improve prediction reliability. The predictive model may be, for example, convolutional neural networks (CNNs), recurring neural networks (RNNs), long short-term memory networks (LSTMs), decisions trees, k-nearest neighbor algorithm configured to generate predictive outputs based on a plurality of historical audio signals, etc. In embodiments, the AI module 250 includes a multi-modal machine learning model configured to identify correlations between at least one bodily-need data, at least one audio signal, and at least one of a heart rate or oxygen level data.

In some embodiments, the predictive module 260 (the predictive model) is configured to generate predictive bodily-needs when the sensor module 230 receives new audio data, movement data, heart rate data, and oxygen data. The predictive model 260 may be configured to process new audio data, movement data, heart rate data, and oxygen data in a manner similar to the data on which the predictive model was generated, by applying any necessary preprocessing steps, such as normalization or encoding. The predictive module 260 then feeds the processed input (the new audio data, movement data, heart rate data, and oxygen data) into its internal structure, such as a set of mathematical equations, decision trees, or neural network layers, depending on the algorithm used (such as the aforementioned CNNs, RNNs, LSTMs, decisions trees, etc.). The predictive module 260 calculates the likelihood or output value based on the learned weights, coefficients, or parameters that were optimized during training. In this way, the predictive module 260 outputs, as a prediction, at least one second bodily-need based on the at least one new second audio signal or new second movement data is received by the wearable device containing the sensor module 230.

In some embodiments, the communication module 202 is configured for receiving, processing, and transmitting a user command and/or one or more data streams. In such embodiments, the communication module 202 performs communication functions between various devices, including the user computing device 145 of FIG. 1, the administrator computing device 185 of FIG. 1, and a third-party computing device 195 of FIG. 1. In some embodiments, the communication module 202 is configured to allow one or more users of the system, including a third-party, to communicate with one another. In some embodiments, the communications module 202 is configured to maintain one or more communication sessions with one or more servers, the administrative computing device 185 of FIG. 1, and/or one or more third-party computing device(s) 195 of FIG. 1. In some embodiments, the communication module 202 may allow users and administrators to communicate with one another.

In some embodiments, a database engine 204 is configured to facilitate the storage, management, and retrieval of data to and from one or more storage mediums, such as the one or more internal databases described herein. In some embodiments, the database engine 204 is coupled to an external storage system. In some embodiments, the database engine 204 is configured to apply changes to one or more databases. In some embodiments, the database engine 204 comprises a search engine component for searching through thousands of data sources stored in different locations.

The user module 212 may store user preferences including the user account information, historical usage data, user personal information, and the like. The user module 212 may facilitate the creation of user's profiles for users, administrators, and others.

In some embodiments, the display module 216 is configured to display one or more graphic user interfaces, including, e.g., one or more user interfaces. In some embodiments, the display module 216 is configured to temporarily generate and display various pieces of information in response to one or more commands or operations. The various pieces of information or data generated and displayed may be transiently generated and displayed, and the displayed content in the display module 216 may be refreshed and replaced with different content upon the receipt of different commands or operations in some embodiments. In such embodiments, the various pieces of information generated and displayed in a display module 216 may not be persistently stored. The display module 216 displays information, notifications, and alerts to the user device which can be viewed and acknowledged by the user. In embodiments, the system is configured to communicates predictive notifications to the user computing device 145 based on the predictive module 260. For example, if the predictive module 260 determine that a bodily-need is food, a notification displaying a “food” or “eating” symbol may be communicated to the user computing device 145.

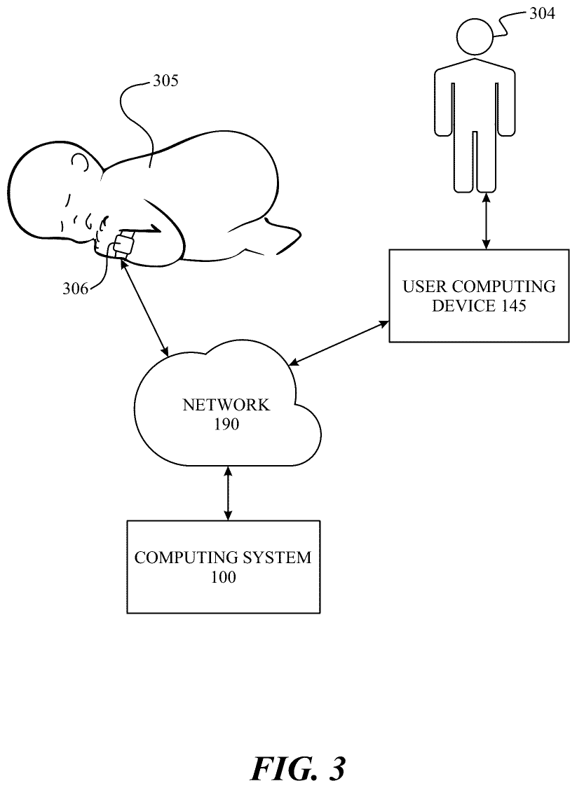

FIG. 3 illustrates a block diagram of an AI-based infant language translation system including a computing system 100, depicted as computing system 100 in FIG. 2, running the application program 200 of FIGS. 1 and 2, and in operable communication with a user computing device 145 and wearable device 306 over a network 190. The user computing device 145 corresponds to the user computing device 145 of FIG. 1, and is operated by user 304, which may be, for example, a parent or caretaker, of infant 305 wearing the wearable device 306. Wearable device 306 contains all or a portion of the sensor module 230 of FIG. 2 such that audio data, movement data, heart rate data, and oxygen data may be passively collected from the infant 305 and communicated over the network 190 to both the user computing device 145 and the computing system 100.

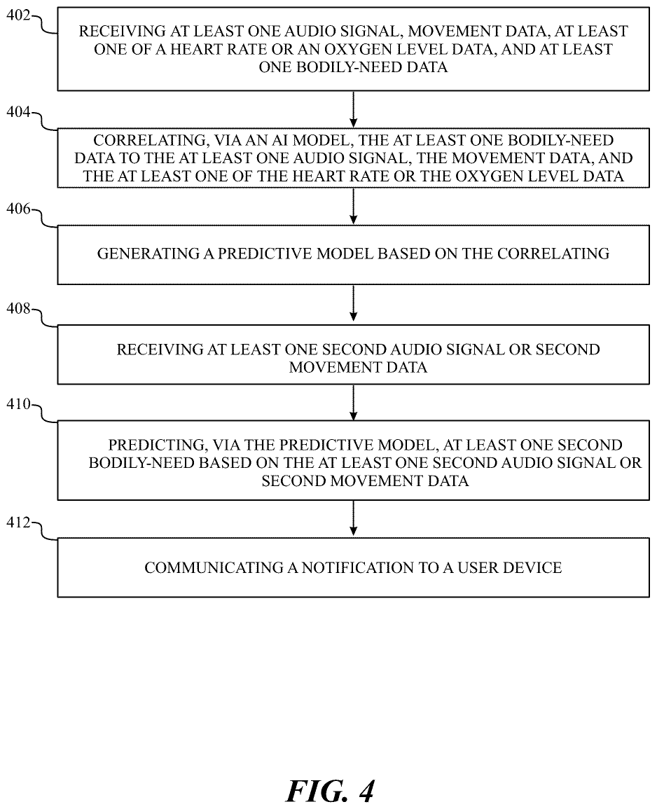

FIG. 4 illustrates a flowchart of an AI-based infant language translation method. In step 402, the method may include receiving at least one audio signal via the sensor module 230 of FIG. 2. In step 404, the method may include receiving movement data via the sensor module 230 of FIG. 2 In step 406, the method may include receiving at least one of a heart rate or an oxygen level data via the sensor module 230 of FIG. 2. In step 408, the method may include receiving at least one bodily-need data via the user module 212 of FIG. 2. In step 410, the method may include correlating the at least one bodily-need data to the at least one audio signal, the movement data, and the at least one of the heart rate or the oxygen level data via the AI module 250 of FIG. 2. In step 412, the method may include generating a predictive model based on the correlation via the AI module 250 of FIG. 2. In step 414, the method may include receiving at least one second audio signal or second movement data via the sensor module 230 of FIG. 2. In step 416, the method may include predicting at least one second bodily-need based on the at least one second audio signal or second movement data via the predictive module 260 of FIG. 2. In step 418, the method may include communicating a notification to a user device via the communication module 202 of FIG. 2.

In this disclosure, the various embodiments are described with reference to the flowchart illustrations and/or block diagrams of methods, apparatus (systems), and computer program products. Those skilled in the art would understand that each block of the flowchart illustrations and/or block diagrams, and combinations of blocks in the flowchart illustrations and/or block diagrams, can be implemented by computer readable program instructions. The computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions or acts specified in the flowchart and/or block diagram block or blocks. The computer readable program instructions can be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions can be loaded onto a computer, other programmable data processing apparatus, or other device to cause a series of operational acts to be performed on the computer, other programmable apparatus, or other device to produce a computer implemented process, such that the instructions that execute on the computer, other programmable apparatus, or other device implement the functions or acts specified in the flowchart and/or block diagram block or blocks.

In this disclosure, the block diagrams in the Figures illustrate the architecture, functionality, and operation of possible implementations of systems, methods, and computer program products according to the various embodiments. Each block in the flowchart or block diagrams can represent a module, segment, or portion of instructions, which comprises one or more executable instructions for implementing the specified logical function(s). In some embodiments, the functions noted in the blocks can occur out of the order noted in the Figures. For example, two blocks shown in succession can, in fact, be executed concurrently or substantially concurrently, or the blocks can sometimes be executed in the reverse order, depending upon the functionality involved. In some embodiments, each block of the block diagrams and/or flowchart illustration, and combinations of blocks in the block diagrams and/or flowchart illustration, can be implemented by a special purpose hardware-based system that performs the specified functions or acts or carry out combinations of special purpose hardware and computer instructions.

In this disclosure, the subject matter has been described in the general context of computer-executable instructions of a computer program product running on a computer or computers, and those skilled in the art would recognize that this disclosure can be implemented in combination with other program modules. Generally, program modules include routines, programs, components, data structures, etc., that perform particular tasks and/or implement particular abstract data types. Those skilled in the art would appreciate that the computer-implemented methods disclosed herein can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated embodiments can be practiced in distributed computing environments where tasks are performed by remote processing devices that are linked through a communications network. Some embodiments of this disclosure can be practiced on a stand-alone computer. In a distributed computing environment, program modules can be located in both local and remote memory storage devices.

In this disclosure, the terms “component,” “system,” “platform,” “interface,” and the like, can refer to and/or include a computer-related entity or an entity related to an operational machine with one or more specific functionalities. The disclosed entities can be hardware, a combination of hardware and software, software, or software in execution. For example, a component can be a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As another example, a component can be an apparatus that provides specific functionality through electronic components without mechanical parts, wherein the electronic components can include a processor or other means to execute software or firmware that confers at least in part the functionality of the electronic components. In some embodiments, a component can emulate an electronic component via a virtual machine, e.g., within a cloud computing system.

The phrase “application” as is used herein means software other than the operating system, such as Word processors, database managers, Internet browsers and the like. Each application generally has its own user interface, which allows a user to interact with a particular program. The user interface for most operating systems and applications is a graphical user interface (GUI), which uses graphical screen elements, such as windows (which are used to separate the screen into distinct work areas), icons (which are small images that represent computer resources, such as files), pull-down menus (which give a user a list of options), scroll bars (which allow a user to move up and down a window) and buttons (which can be “pushed” with a click of a mouse). A wide variety of applications is known to those in the art.

The phrases “Application Program Interface” and API as are used herein mean a set of commands, functions and/or protocols that computer programmers can use when building software for a specific operating system. The API allows programmers to use predefined functions to interact with an operating system, instead of writing them from scratch. Common computer operating systems, including Windows, Unix, and the Mac OS, usually provide an API for programmers. An API is also used by hardware devices that run software programs. The API generally makes a programmer's job easier, and it also benefits the end user since it generally ensures that all programs using the same API will have a similar user interface.

The phrases “computing device” or “central processing unit” as is used herein means a computer hardware component that executes individual commands of a computer software program. It reads program instructions from a main or secondary memory, and then executes the instructions one at a time until the program ends. During execution, the program may display information to an output device such as a monitor.

The term “execute” as is used herein in connection with a computer, console, server system or the like means to run, use, operate or carry out an instruction, code, software, program and/or the like.

In this disclosure, the descriptions of the various embodiments have been presented for purposes of illustration and are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein. Thus, the appended claims should be construed broadly, to include other variants and embodiments, which may be made by those skilled in the art.

It will be appreciated by persons skilled in the art that the present embodiment is not limited to what has been particularly shown and described hereinabove. A variety of modifications and variations are possible considering the above teachings without departing from the following claims.

I/We claim:

1. A computer-implemented method comprising:
receiving, via a computing device, at least one audio signal;
receiving, via the computing device, movement data;
receiving, via the computing device, at least one of a heart rate or an oxygen level data;
receiving, via the computing device, at least one bodily-need data;
correlating, via the computing device implementing an artificial intelligence (AI) model, the at least one bodily-need data to the at least one audio signal, the movement data, and the at least one of the heart rate or the oxygen level data;
generating, via the computing device implementing the AI model, a predictive model based on the correlating;
receiving, via the computing device, at least one second audio signal or second movement data;
predicting, via the computing device implementing the predictive model, at least one second bodily-need based on the at least one second audio signal or second movement data; and
communicating, via the computing device, a notification to a user device including the second bodily-need.

2. The computer-implemented method of claim 1, wherein the receiving the at least one audio signal comprises passively measuring audio via a microphone of a wearable device in operable communication with the computing device.

3. The computer-implemented method of claim 1, wherein the receiving the at least one of the heart rate or the oxygen level data comprises measuring the least one of the heart rate or the oxygen level via a sensor in operable communication with the computing device, wherein the sensor is in operative contact with a human.

4. The computer-implemented method of claim 1, wherein the receiving the at least one bodily-need data comprises receiving text input via the computing device.

5. The computer-implemented method of claim 1, wherein the AI model comprises a multi-modal machine learning model configured to identify correlations between the at least one bodily-need data, the at least one audio signal, and the at least one of the heart rate or the oxygen level data.

6. The computer-implemented method of claim 1, wherein the predictive model comprises a k-nearest neighbor algorithm configured to generate predictive outputs based on a plurality of historical audio signals.

7. The computer-implemented method of claim 1, wherein the audio signal is a noise from a baby.

8. The computer-implemented method of claim 1, wherein the predicting the at least one second bodily-need is based on the at least one second audio signal and a plurality of historical audio signals from a baby.

9. The computer-implemented method of claim 1, wherein the bodily-need data comprises information relating to at least one of sleep, nutrition, weight, height, or temperature data.

10. A system comprising:
at least one computing device in operable communication with a network;
an application server in operable communication with the at least one computing device over the network, the application server configured to host an application program configured to:
receive at least one audio signal;
receive movement data;
receive at least one of a heart rate or an oxygen level data;
receive at least one bodily-need data;
correlate, via an artificial intelligence (AI) model, the at least one bodily-need data to the at least one audio signal and the at least one of the heart rate, the movement data, or the oxygen level data;
generate, via the AI model, a predictive model based on the correlating;
receive at least one second audio signal or second movement data; and
predict, via the predictive model, at least one second bodily-need based on the at least one second audio signal or second movement data.

11. The system of claim 10, wherein the receiving the at least one audio signal comprises passively measuring audio via a microphone of a wearable device in operable communication with the computing device.

12. The system of claim 10, wherein the receiving the at least one of the heart rate or the oxygen level data comprises measuring the least one of the heart rate or the oxygen level via a sensor in operable communication with the computing device, wherein the sensor is in operative contact with a human.

13. The system of claim 10, wherein the receiving the at least one bodily-need data comprises receiving text input via the computing device.

14. The system of claim 10, wherein the AI model comprises a multi-modal machine learning model configured to identify correlations between the at least one bodily-need data, the at least one audio signal, and the at least one of the heart rate or the oxygen level data.

15. The system of claim 10, wherein the predictive model comprises a k-nearest neighbor algorithm configured to generate predictive outputs based on a plurality of historical audio signals.

16. The system of claim 10, wherein the audio signal is a noise from a baby.

17. The system of claim 10, wherein the predicting the at least one second bodily-need is based on the at least one second audio signal and a plurality of historical audio signals from a baby.

18. The system of claim 10, wherein the bodily-need data comprises information relating to at least one of sleep, nutrition, weight, height, or temperature data.

19. A software product comprising at least one computer readable storage media having application instructions collectively stored on the at least one computer readable storage media, the application instructions executable to:
receive at least one audio signal;
receive movement data;
receive at least one of a heart rate or an oxygen level data;
receive at least one bodily-need data;
correlate, via an artificial intelligence (AI) model, the at least one bodily-need data to the at least one audio signal and the at least one of the heart rate, the movement data, or the oxygen level data;
generate, via the AI model, a predictive model based on the correlating;
receive at least one second audio signal or second movement data; and
predict, via the predictive model, at least one second bodily-need based on the at least one second audio signal or the second movement data.
